(19) United States
(12) Patent Application Publication
(10) Pub. No.: US 2019/0343631 A1
(43) Pub. Date: Nov. 14, 2019

(54) CIRCUMFERENTIALLY CONSTRICTIVE ANNULOPLASTY RING

(71) Applicant: Northwestern University, Evanston, IL (US)

(72) Inventor: Patrick M. McCarthy, Chicago, IL (US)

(73) Assignee: Northwestern University, Evanston, IL (US)

(21) Appl. No.: 16/421,998

(22) Filed: May 24, 2019

Related U.S. Application Data
(63) Continuation-in-part of application No. 15/735,128, filed on Dec. 8, 2017.
(60) Provisional application No. 62/241,664, filed on Oct. 14, 2015.
(60) Provisional application No. 62/172,722, filed on Jun. 8, 2015.
(60) Provisional application No. 62/676,580, filed on May 25, 2018.

(51) Int. Cl. A61F 2/24

(52) U.S. Cl. CPC A61F 2/2448; A61F 2220/0008; A61F 2250/001; A61F 2210/0057

(57) ABSTRACT

An annuloplasty ring including a spring-like inner element that is able to be stretched to accept a prosthetic valve that, when stretched, places a circumferential constrictive force on the valve to prevent paravalvular leakage.

### RELATED APPLICATIONS

This application is a Continuation-in-part of U.S. application Ser. No. 15/735,128 filed Dec. 8, 2017 entitled Annuloplasty Ring For Receiving A Replacement Valve and claims benefit of and priority to U.S. Provisional Application Ser. No. 62/676,580 filed May 25, 2018 entitled Circumferentially Constrictive Annuloplasty Ring, both of which are hereby incorporated herein by reference in their entireties.

### FIELD OF THE INVENTION

The present invention relates to treatments for heart valve regurgitation, including mitral valve disease and triscupid valve disease, and may be applicable for aortic and pulmonic valve disease.

### BACKGROUND OF THE INVENTION

Annuloplasty rings and various methods of implanting such rings for the treatment of heart valve disorders has been known for several years and represents a significant advancement in the treatment of heart disease. Annuloplasty rings are sutured, clipped, or otherwise secured to the patient's valve annulus and are used to reduce the diameter of an enlarged or diseased heart valve, thereby allowing the valve leaflets to establish or reestablish coaptation, thereby reducing or eliminating regurgitant flow through the valve.

However, it has been observed in some patients that although the ring properly treats the diseased valve, the valve condition continues to worsen and thus necessitates a valve replacement. Historically such valve replacements have required another open-heart surgery including first the excision of the annuloplasty ring followed by the implantation of a new valve. Although this multi-step process has proved useful and has saved and prolonged the lives of numerous patients, the excision and implantation process is time consuming and challenging for the operating physician, and potentially very dangerous for the patient. For example, if the condition of the valve and surrounding tissue is particularly diseased, the excision of the ring can often times render it very difficult to find suitable tissue to securely attach the new replacement valve.

The necessity of excising the ring at open heart surgery arises from the rigid, and/or non-resilient, non-conformable nature of existing rings. The ring also constricts the orifice of the valve and may not allow a properly sized prosthetic valve to be placed inside the ring. The ring also may distort the prosthetic valve causing a regurgitant leak within the prosthetic valve. The shape and size of the ring may not correspond well to the prosthetic valve and may lead to a regurgitant leak between the ring and the prosthetic valve called paravalvular regurgitation. Most rings are roughly D shaped and almost all prosthetic valve replacements are round so these spatial relationships have made the practical application of placing a valve inside a ring very difficult.

As a result, there is a distinct need for an annuloplasty ring that provides both an initial successful treatment of a diseased valve (e.g., treatment of valve regurgitation) and then provides, at a later date, a platform for receiving a replacement valve, should that patient's valve continue to deteriorate and require replacement. Such an annuloplasty ring would not require excision in such a circumstance, but, rather, would offer characteristics that enable placement and fixation of a prosthetic heart valve within the previously implanted annuloplasty ring.

### OBJECTS AND SUMMARY OF THE INVENTION

The present invention is directed to meeting the aforementioned need for an annuloplasty ring that provides both an initial successful treatment of a diseased valve and a platform for receiving a replacement valve, sometimes referred to herein as a “Valve In Ring” or “VIR” procedure. The present invention accomplishes this by providing an annuloplasty ring that stretches and deforms around a prosthetic valve and then squeezes the prosthetic valve like a rubber band once released.

More specifically, the ring of the present invention that has an elongate core, surrounded by one or more layers, and having a shape configured to establish an optimal perimeter for a human blood valve when implanted in a circulatory system and including at least one elastic feature allowing the core to be stretched from a resting state to a stretched state, and wherein the core is biased toward said resting state. The ring is implanted in a resting state designed to optimize performance of the patients own valve and the elastic nature of the ring also allows the ring to stretch in reaction to blood pressure forces and changes in blood flow. This allows the ring to more closely mimic native valve behavior as well as prevents undesired tissue stresses to be concentrated on areas surrounding ring fixation mechanisms such as sutures and the like. The stretching characteristic of the ring allows the ring to be stretched during valve in ring implantation such that it places a squeezing force on the native valve annulus that promotes long-term coaptation of the annulus to the prosthetic valve.

The squeezing characteristics of the elastic ring makes the ring stretchable and deformable, and therefore uniquely suited to receiving a prosthetic valve, such as an implanted transcatheter valve (TCV), a “sutureless valve” or other prosthesis. Due to the squeezing effect of the elastic, the ring functions as a gasket or rubber band around the prosthetic valve. The squeezing causes the ring to seal around the valve, thereby preventing paravalvular leakage.

One aspect of the invention provides a ring with an incorporated elastic core element designed to impart inward radial force when a valve is inserted within the ring. The valve may be a transcatheter valve or other less-invasive approaches and valves, or may be valves implanted via open heart surgery.

In another aspect of the invention, the inner element takes the form of a spring. The spring may be a variety of configurations. Non-limiting examples include: open coil, closed coil, helical, non-helical, and double-helical.

In another aspect of the invention, the characteristics of the inner element are designed to exert a circumferential force, either uniform or non-uniform, which can be determined by the properties of the spring including the metal, alloy, or other material, the winding of the spring, and other design and manufacturing elements.

The amount of radial force exerted by the ring will be designed according to the various sizes of the rings and the intended size of transcatheter valve implanted within. For example, the winding or other properties of a 28 mm ring accommodating a 26 transcatheter valve will be different than a 34 mm ring accommodating a 29 mm transcatheter valve. But both will be designed to create a certain “snug” uniform inward force that will be within a range of radial force for a given transcatheter valve.

Other aspects of the ring are also designed to stretch and impart inward radial force including the material surrounding the inner element (such as silicone), and the covering of the ring, such as Dacron.

One aspect of the invention provides a complete ring with an incomplete inner element. In other words, the inner element does not extend the entirety of the ring.

In another aspect, both the ring and the inner element are continuous.

In yet another aspect, neither the ring nor the inner element are continuous.

These various configurations may have advantages based on the targeted valve, for example, the mitral or tricuspid valve. The optimal configuration may also be dependent on the unique age and condition of the targeted heart valve.

In one aspect, anchoring mechanisms, in addition to customary surgical fixation, may be used in part or along the length of the ring to aid in fixation when the ring is activated (stretched) by the transcatheter valve

In another aspect, the ring may be modified by: incorporating anchoring elements to attach it to the valve annulus; accommodating anchoring elements surrounding the ring; and/or making it discontinuous to allow a percutaneous, transcatheter, or other less invasive implantation.

The properties of the ring to impart inward radial and/or circumferential force when stretched by a transcatheter valve remain even if it is implanted by a transcatheter approach and anchored in a fashion that is different than an open heart surgical approach.

The ring may be manufactured with a memory metal or alloy, such as Nitinol, that allows the metal to change shape when activated by heat or other energy forces, such as ultrasound or electrical current, for example.

The present invention also includes a method of treating or replacing a diseased heart valve. Initially, annuloplasty procedure is performed by installing an annuloplasty ring of the present invention around a regurgitant valve. The annuloplasty ring reestablishes coaptation of the native valve leaflets. If the native valve leaflets or surrounding structures become diseased later and it is determined that the diseased valve needs to be replaced, the method involves placing a prosthetic valve; using a transcatheter valve, sutureless valve, or through open heart surgery, within the installed annuloplasty ring. This is done without removing the ring and with or without removing the native heart valve. The prosthetic valve may be sewn or otherwise attached directly to the ring. The ring thus provides an ideally-shaped platform for receiving a prosthetic heart valve.

The present invention provides a ring that may be secured to the valve annulus via open surgery or percutaneously. Percutaneous approaches for implanting annuloplasty rings are currently under development. A percutaneous approach developed by Valtech Cardio of Or Yehuda, Israel, and called the Cardioband, provides an example and is incorporated by reference herein. More information is provided at http:/www.valtechcardio.com. An embodiment of the device and method is shown and described in U.S. Pat. No. 8,715,342 to Zipory et al. and entitled, Annuloplasty Ring With Intra-Ring Anchoring, filed on May 7, 2009 and incorporated by reference herein. However, the Cardioband is not elastic and is not designed for receiving a prosthetic heart valve. The elastic nature of the ring, and especially the incomplete embodiments, make the ring especially suited for transcatheter delivery as well as accepting a subsequent prosthetic valve.

Another example, developed by Edwards Lifesciences Corp. of Irvine Calif. is described in U.S. Pat. No. 8,287,591 to Keidar et al. and entitled Transformable Annuloplasty Ring Configured to Receive a Percutaneous Prosthetic Heart Valve Implantation, filed on Sep. 19, 2008 and incorporated by reference herein. However, this device requires a balloon to expand the device in order to receive a prosthetic valve.

Still another example is described in U.S. Pat. No. 6,187,040 to Wright, entitled Mitral And Tricuspid Annuloplasty Rings. Wright uses a coil within the ring but it is not elastic. Rather, the coil defines a lumen through which a string is routed. The string is tightened during implantation for purposes of sizing.

One skilled in the art will understand that the methods and ring embodiments of the present invention are novel and advantageous for several reasons, only a few of which include being designed for VIR procedures, providing optimal performance characteristics such as flexibility, deformability, radiopacity, and the obviation of pre-balloon procedures. Moreover the embodiments of the rings of the present invention, while being novel, provide a familiar appearance designed to optimize ease of use with a shallow learning curve for the implanting surgeon. The ring designs are also relatively easy to manufacture.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a perspective view of an embodiment of a ring of the invention having been placed at a target mitral valve site;

FIG. 2 is a plan view of an embodiment of an inner element of the invention;

FIG. 3 is a perspective view of an embodiment of a ring of the invention having been placed at a target valve site and having been expanded with the implantation of a prosthetic valve therein;

FIG. 4 is a perspective view of an embodiment of a ring of the invention implanted at a valve site; and,

FIG. 5 is a is a perspective view of an embodiment of a ring of the invention implanted at a valve site.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

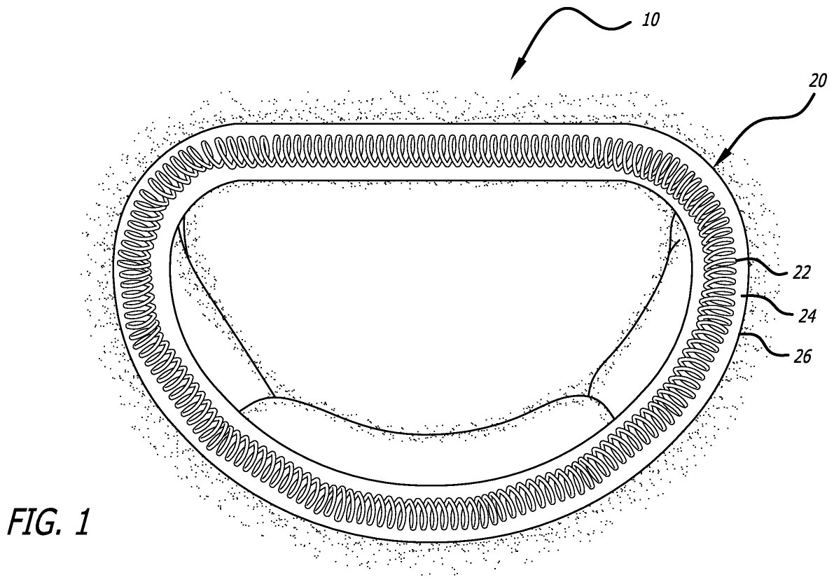

Referring now to the figures, and first to FIG. 1, there is shown an embodiment 20 of an annuloplasty ring 10 of the invention. The ring 20 has a D shape, for use with a mitral valve, and generally includes an elongate core 22, also referred to herein as an inner elastic element, that may be in the form of a spring, as explained in greater detail below.

The inner element 22 is covered by one or more layers. The embodiment shown in FIG. 1 includes a silicone layer 24, which is surrounded by an outer fabric layer 26. The silicone layer 24 ensures a smooth transition of the inner element 22 from a relaxed configuration, such as when used in an annuloplasty capacity, to a stretched configuration, such as when accommodating a prosthetic valve. The outer fabric layer 26 is suitable for accepting sutures or other anchoring mechanisms that secure the ring 10 to the annulus of the native valve. For example, the covering material may be that which is common in the art, such as a double velour fabric or other Dacron fabric. Such a fabric assists in anchoring by providing a medium for receiving sutures and also promoting in-growth of tissue. Additionally, depending on the performance of the selected core material, the need for a cover may be obviated.

The inner element 22 is shown as a single helical, open-coil spring. The purpose of the inner element 22 is to place a constrictive radial force on a prosthetic valve when the ring 10 is stretched to accommodate the prosthetic valve. All of the various ring embodiments described herein include an inner elastic element that give the ring an elastic stretchability from a resting state to a stretched state. Though the ring is implanted in its resting state, the ring is flexible and allows for normal stretching and function of the valve. When the ring is in the stretched state, a squeezing force is imparted on the object or forces, such as blood pressure, that are placing the device in the stretched state. The squeezing characteristics of the elastic ring makes the ring both stretchable and deformable, and therefore uniquely suited to receiving a prosthetic valve, such as an implanted transcatheter valve (TCV) or other prosthesis. Due to the squeezing effect of the inner element, the ring and the annular tissue to which it is fixed function as a gasket or rubber band around the prosthetic valve. The squeezing causes the ring and tissue to seal around the valve, thereby preventing paravalvular leakage and reducing the risk that the valve may move, migrate, or embolize.

As such, with this purpose in mind, one skilled in the art will understand that the inner elastic element 22 may take the forms of other elastic mechanisms without departing from the spirit of the invention. For example, a single-helical closed-coil spring may be used. Other examples include, but are not limited to, elastomeric bands, extension springs, barrel springs magazine springs, garter springs, conical springs, wire rings, Nitinol elements, to name just a few. The springs may be constructed of biocompatible materials including, but not limited to, Nitinol or an alloy, stainless steel, cobalt chromium, titanium, nickel or others.

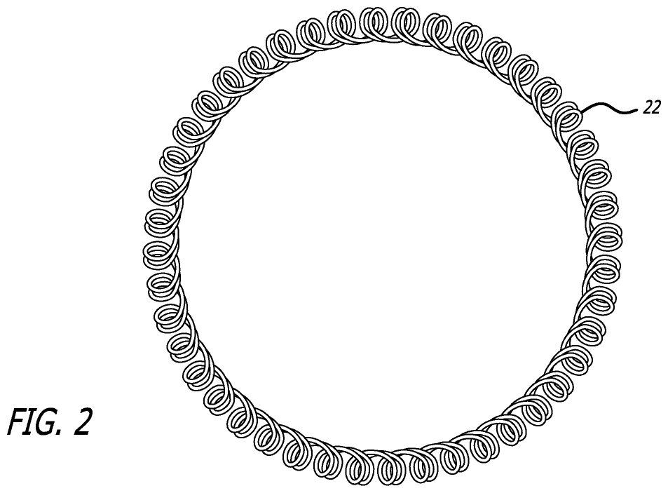

FIG. 2, for example, shows an inner element 22 in the form of a double-helical open-coil spring. The double-helical open-coil spring is formed by nesting two open-coil helical springs together. It is envisioned that various embodiments are provided such that a physician is able to select a ring that best suits the individual needs of the patient.

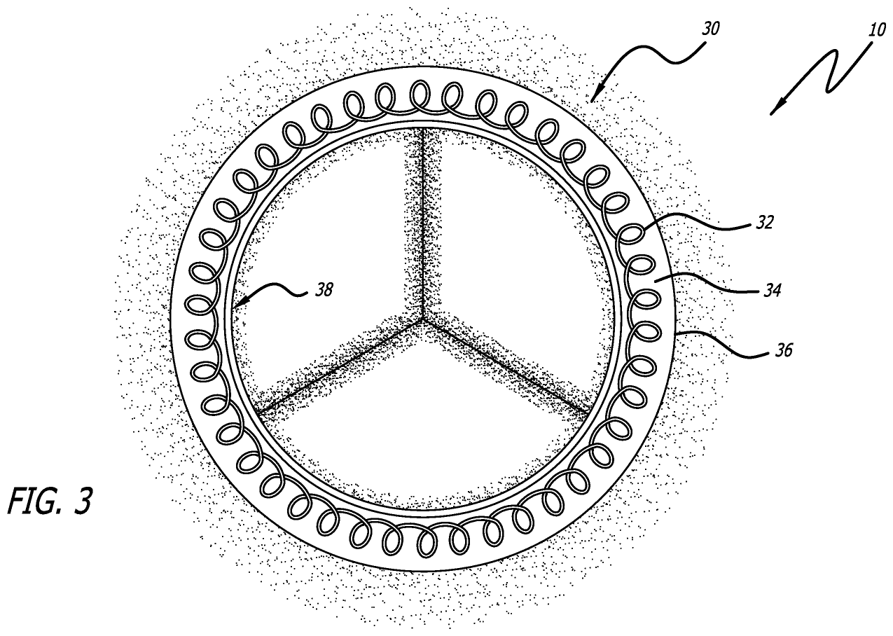

FIG. 3 shows an embodiment 30 of a ring 10 that is suited for use with a mitral or tricuspid valve. The embodiment 30 includes a complete-loop inner element 32, a complete first layer 34 and a complete second layer 36. The embodiment 30 is shown in an expanded condition surrounding a prosthetic valve 38. In this expanded condition the inner element 32 is placing a circumferential constrictive force on the prosthetic valve 38.

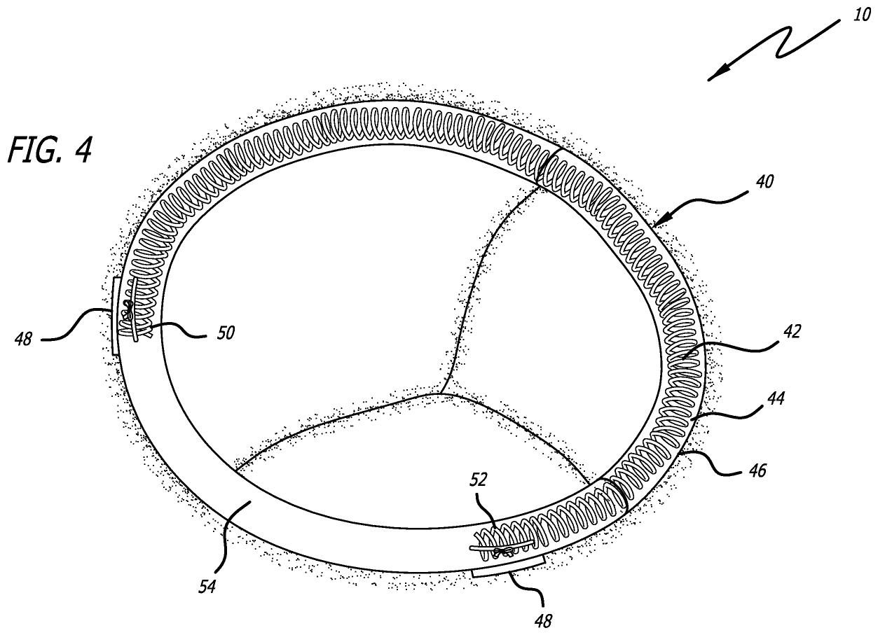

FIG. 4 shows an embodiment 40 of a ring 10 that includes an incomplete inner element 42 contained within complete layers 44 and 46. The embodiment 40 also includes a pair of anchor pads 48. The anchor pads 48 are located under a site of reinforced suturing and are designed to spread the forces of the sutures out over a wider area. The pads 48 are located at the ends 50 and 52 of the inner element 42.

The incomplete inner element 42 defines a gap 54 between the pads 48 and the ends 50 and 52, characterized by layers 44 and/or 46 but without an inner element 42. This gap 54 is designed to be aligned with an area of the valve annulus that includes a nerve bundle that carries cardiac conduction signals. By avoiding the placement of an inner element in this location, interference with this nerve traffic and the heart rhythm is avoided.

The inclusion of the layers 44 and/or 46 in the area of the gap 54 may serve to aid in focusing or controlling the contractile nature of the inner element 42, and may promote ingrowth to serve as a gentle, non-sutured anchor in this area of increased nerve-traffic. These layers may also assist in preventing paravalvular leakage in this area when a prosthetic valve is installed.

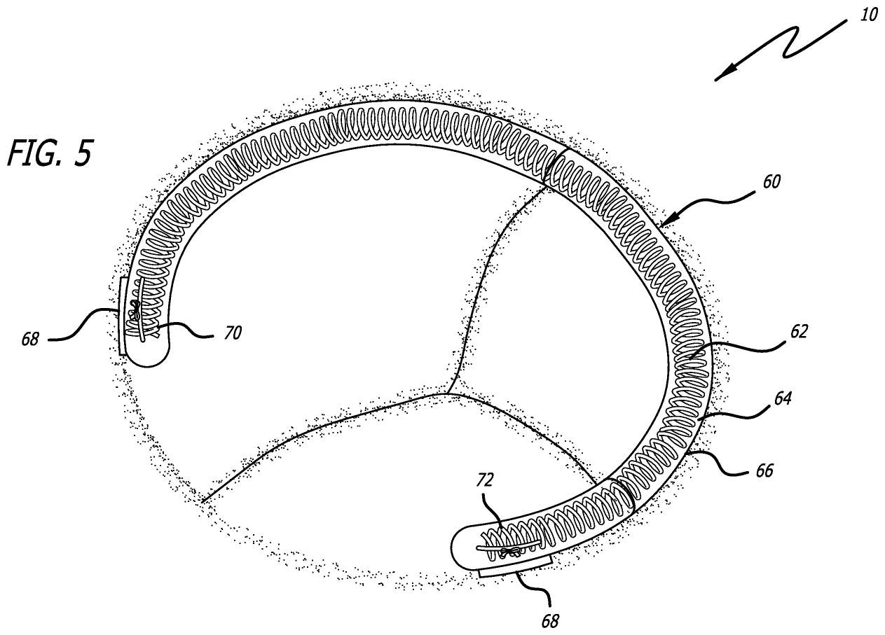

FIG. 5 shows an alternative embodiment 60 of a ring 10 that does not include fabric spanning the gap of the incomplete element. Like embodiment 40, ring 60 includes an incomplete inner element 62 contained within complete layers 64 and 66. The embodiment 60 also includes a pair of anchor pads 68. The anchor pads 68 are located under a site of reinforced suturing and are designed to spread the forces of the sutures out over a wider area. The pads 68 are located at the ends 70 and 72 of the inner element 62.

The incomplete inner element 62 defines an open gap 74 between the pads 68 and the ends 70 and 72. The layers 64 and 66 terminate at the ends 70 and 72. As such, embodiment 60 is an incomplete ring. This gap 74 is designed to be aligned with the with the cardiac conduction system and septum.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An annuloplasty ring comprising:
an inner element;
at least one layer surrounding the inner element;
wherein said inner element is elastic such that when expanded from a relaxed configuration to a stretched configuration by an object, the inner element exerts a constrictive radial force on the object.

2. The annuloplasty ring of claim 1 wherein said inner element comprises a spring.

3. The annuloplasty ring of claim 1 wherein said inner element comprises a complete circle.

4. The annuloplasty ring of claim 1 wherein said at least one layer comprises a fabric outer layer.

5. The annuloplasty ring of claim 4 wherein said at least one layer comprises a silicone layer between said inner element and said fabric outer layer.

6. The annuloplasty ring of claim 1 wherein said inner element comprises a double helical spring.

7. The annuloplasty ring of claim 1 wherein said inner element comprises a single helical spring.

8. The annuloplasty ring of claim 1 wherein said inner element comprises an incomplete loop.

9. The annuloplasty ring of claim 1 wherein in said relaxed state said inner element comprises a D-shape and in said stretched state said inner element comprises at least a partial loop.

10. The annuloplasty ring of claim 1 wherein said inner element comprises an incomplete loop and said at least one layer comprises an outer layer that is a complete loop.

11. The annuloplasty ring of claim 1 wherein said inner element comprises an incomplete loop and said at least one layer comprises an incomplete loop.

12. The annuloplasty ring of claim 1 further comprising anchor pads.

13. A method of replacing a heart valve having an installed annuloplasty ring comprising: installing a prosthetic heart valve within said annuloplasty ring while elastically stretching said ring, said annuloplasty ring including an inner element that places a constrictive forced on the prosthetic valve.

14. The method of claim 13 wherein said inner element places a circumferential constrictive force on the prosthetic valve.

15. A method of treating a diseased heart valve comprising:
implanting an annuloplasty ring around a diseased heart valve to cause leaflets of said diseased heart valve to coapt, said annuloplasty ring including at least one elastic inner element allowing the ring to be stretched from a resting state to a stretched state, and wherein said ring is biased toward said resting state;
installing a prosthetic valve within said ring if said native valve relapses into a diseased state.

16. The method of claim 15 wherein said inner element comprises a spring.

17. The method of claim 16 wherein said inner element comprises a single helical spring.

18. The method of claim 15 wherein said inner element comprises a double helical spring.

19. The method of claim 18 wherein said inner element extends along a substantial entirety of said elongate core.

20. The method of claim 15 wherein said ring further includes at least one layer surrounding said inner element.
